(19) United States
(12) Patent Application Publication
(10) Pub. No.: US 2023/0273057 A1
(43) Pub. Date: Aug. 31, 2023

(54) Ultrasonic Gas Flow Calibration Device

(71) Applicant: USCOM Limited, Sydney, New South Wales (AU)

(72) Inventors: Robert Allan Phillips, Coffs Harbour, New South Wales (AU); George F. Ferenczi, Macleod, Victoria (AU)

(21) Appl. No.: 18/040,593

(22) PCT Filed: Aug. 3, 2021
(86) PCT No.: PCT/AU2021/050842

§ 371 Date: Feb. 3, 2023

(30) Foreign Application Priority Data
Aug. 3, 2020 (AU) 2020902715

(51) Int. Cl. G01F 1/667; G01F 1/66; G01F 25/10; A61M 16/00

(52) U.S. Cl. CPC G01F 1/667; G01F 1/662; G01F 25/10; A61M 16/0003; A61M 2209/02; A61M 2205/3334; A61M 2016/0027

(57) ABSTRACT

A method of monitoring the flow of a gas along a channel, the method including the steps of: Utilising at least a first ultrasonic transducer to project an alternating ultrasonic signal substantially transverse to the direction of gas flow and ultrasonic receivers to receive the signals; Sampling the ultrasonic signal after it traverses the gas flow; and Processing the sampled signal to determine properties of the gas and flow parameters relating thereto.

### FIELD OF THE INVENTION

The present invention relates to gas or fluid flow and pressure monitoring, and, includes an improved method and apparatus for evaluation the functionality of ventilators and other mechanical respiratory devices and also provides a method to intervene in their operation to optimise performance.

### BACKGROUND OF THE INVENTION

Any discussion of the background art throughout the specification should in no way be considered as an admission that such art is widely known or forms part of common general knowledge in the field.

Respiration is a vital physiologic function that provides the body with oxygen to maintain the viability of the cells, organs and organism. In many diseases, the respiratory system is effected and requires support. There are a variety of devices, including ventilators, which are specialised to supplement and control respiratory function, and are commonly deployed in critical care medicine. The accuracy of these interventions is essential for effectiveness, and substantially depends on the reliability and accuracy of the performance of the ventilators. While most ventilators have some form of self maintenance functionality and the flow and pressure signals are reset between uses, intermittent high resolution calibration is usually employed periodically as a standard of care.

Example ventilator monitoring systems can be seen from US Patent Publications: 2014/0288456, and 20050034721, both incorporated here by cross reference.

A wide range of ventilators, anaesthesia machines, and mechanical respirators exist with the objective of delivering a controlled supply of gas for safe inspiration and expiration in a variety of iterations. These devices generally feature characteristic functional subsystems. The devices typically include two breathing gas conduits, one for inspiration and one for expiration. The two tubes typically meet in a Y connector on the patient side. The two tubes can be attached to the side of the ventilator device via a connector. The ventilator device typically has a mechanical motor and pump that provides adequate flow of air and other gas to and from the patient. The device is also equipped with one or more flow sensors, typically for both the inspiratory and expiratory circuits, and flow control valves.

Testing, calibration and control of the above complex devices is an increasingly significant concern, as the failure and/or malfunction of these devices can result in impaired patient outcomes including death.

An example which demonstrates the significance of correct maintenance of such devices occurred in New York during the 2019 COVID-19 pandemic where over 90% of the patients on medical ventilators died. This may have been partially attributable to ineffective ventilator operation, flow, volume and pressure control, and calibration.

Current ventilator test devices (anaesthesia machine testers) are designed to perform accurate measurements of gas parameters (see patent CN2585215Y). Existing ventilator testers typically can display the various measured parameters on a small screen and the test operator manually compares these output values with the theoretical set values of the ventilator under test, and then manually re-calibrates the ventilator settings to the test values.

In ventilators, the relevant measured parameters are the volume of the gas generated, the rate of gas flow and the pressure of the gas, as well as CO₂, O₂ and time parameters. Typically, the measurements and sensors of the test system need to be more accurate than similar measurements and sensors the device being tested. U.S. Pat. No. 6,266,995 describes such a system.

A strict requirement of any test device is therefore the accurate measurement of gas flow volume and pressure as well as O₂ and CO₂ across a wide flow range of respiratory outputs as occurs in neonates and in adult athletes or those with respiratory disease. According to the current state of the art, such accurate flow and volume measurement is performed by one or more (usually two) flowmeters with different measurement ranges that can be interchangeably connected to the body of a ventilator tester.

Flow meters used in ventilator (anesthesia machine) test devices are sometimes derived from respiratory flow meters used in other medical technology areas. Such flow sensors include differential pressure measurement, pitot-tube based and thermopile methods are most widely used, but have methodological and accuracy limitations.

One form of a flow and volume parameter measurement apparatus is disclosed in WO 2009/071960A1 entitled Method and Apparatus for Determining the Flow Parameters of a Streaming Medium”, the contents of which are incorporated by cross reference.

### SUMMARY OF THE INVENTION

It is an object of the invention, in its preferred form to provide an improved ultrasonic fluid flow and pressure testing and calibration device. The fluid can include a gas.

In accordance with a first aspect of the present invention, there is provided a method of monitoring the flow of a fluid along a channel, the method including the steps of: utilising at least a first ultrasonic transducer to project an alternating ultrasonic signal substantially transverse to the direction of fluid flow; sampling the ultrasonic signal after it traverses the fluid flow; and processing the sampled signal to determine properties of the fluid and flow parameters relating thereto.

The sampling preferably can include sampling the ultrasonic signal at least two points substantially opposite the first ultrasonic transducer. At least one of the points can be upstream of the first ultrasonic transducer and one can be down stream of the first ultrasonic transducer.

The method can also include simultaneously monitoring the fluid pressure within the channel. The fluid pressure can be monitored at multiple points along the channel. One of the points can be opposite the first ultrasonic transducer opposite the channel. In another embodiment, the pressure signal can be obtained at the end of the flow sensor tube, in order to make sure that the pressure drop inside the apparatus is not affecting the pressure measurement.

In accordance with a further aspect of the present invention, there is provided a device for monitoring the flow of a fluid along a tube, the device including: A first tube having an inlet and outlet for connection to a fluid source and a fluid sink; At least one ultrasonic transducer located on one side of the tube for projecting an ultrasonic signal into the tube substantially transverse to the fluid flow in the tube; At least one ultrasonic sensor located on an opposed side of the tube for monitoring the receipt of the ultrasonic signal on the opposed side of the tube; and Processing means interconnected to the at least one ultrasonic transducer and the at least one ultrasonic sensor for determining flow parameters of the fluid within the tube.

### BRIEF DESCRIPTION OF THE DRAWINGS

Embodiments of the invention will now be described, by way of example only, with reference to the accompanying drawings in which:

FIG. 1 illustrates a schematic sectional view showing the basic operation of the core flow and volume measurement method of the invention;

FIG. 2 illustrates a cross sectional schematic view of a flow tube used in the invention, with optional locations of the pressure signal sampling;

FIG. 3 illustrates a CAD model of a plastic part of one form of implementation of the flow tube of the invention;

FIG. 4 illustrates a side perspective view, partly in section of the arrangement of FIG. 3;

FIG. 5 illustrates a cross sectional schematic view of another embodiment of the flow tube with an integrated pressure sampling tube and basic components for a handheld device including battery;

FIG. 6 illustrates a simplified schematic view of an embodiment of the flow and pressure sensor, in a handheld portable implementation;

FIG. 7 illustrates a detailed 3D rendering of an embodiment, with an attached pressure subsystem which is detachable;

FIG. 8 illustrates a side perspective view of a detailed 3D rending of an embodiment;

FIG. 9 illustrates a photograph of a first functional prototype implementing the invention, connected in a test patient circle of a medical ventilator;

FIG. 10 is a functional block diagram of one form of processing architecture of the invention;

FIG. 11 is a screen shot of one form of user interface of the ventilator tester functional prototype software with specific distinguished characteristic sections;

FIG. 12 is a user interface of the ventilator tester prototype software with a separate display areas for the flow, volume and pressure data;

FIG. 13 illustrates an example display of the flow, pressure and volume data and the synchronization among them, in a way so that the co-processed flow-pressure signals result in a more accurate flow determination.

### DETAILED DESCRIPTION OF THE INVENTION

The preferred embodiments of the present invention are based on combining a digital ultrasonic method for determining the flow and volume fluid/gas parameters with a pressure sensor for monitoring contemporaneous pressure measurements. The current method utilises digital ultrasonic flow monitoring.

The digital ultrasonic method for determining the flow parameters of a gas medium flowing in a tube depends on propagating acoustic compression waves into the tube with ultrasonic transducers. The waves are contemporaneously transmitted obliquely toward the flow, and obliquely against the direction of flow, with the acoustic signal received by sensing transducers positioned on the opposing side of the flow tube. The flow and volume parameters of the flowing medium are obtained from the processing of the parameters of the received acoustic waves.

The apparatus can include a series of ultrasonic transducers and the applied longitudinal (acoustic) wave frequency is between about 40 Khz and 200 Khz.

The ultrasonic flow meter pressure sensor sampling tubes can be recessed into and passing through the wall of the flow tube. The pressure sampling tube stops in line with the plane of the inner surface of the flow tube, while the actual pressure sensor is located outside the wall of the tube.

The ultrasonic flow sensor and the integrated pressure sensor transmit electronic signals to a computer or a microcontroller in a handheld device where they are decoded by processing software and forwarded to a display showing precision flow, pressure and volume values in real time or near real time. In some embodiments, the ultrasonic flow sensor and the integrated pressure sensor transmit electronic signals to a handheld device where a processing firmware displays precision flow, pressure and volume values in real time or near real time.

In some embodiments, the flow and pressure sensors and the handheld device can be combined in an integral unit.

In some embodiments, the integral ventilator test unit can also transmit data signals to a computer via a data cable or in a wireless manner so the ventilation data can also be displayed by the computer software in real time.

In some embodiments, the flow, volume and pressure data measured and processed by the handheld ventilator tester device and transmitted to the computer software, are used for direct feedback in the medical ventilator, in a way so that the actual values of the ventilator are adjusted by automatically calculated offsets and linearization parameters.

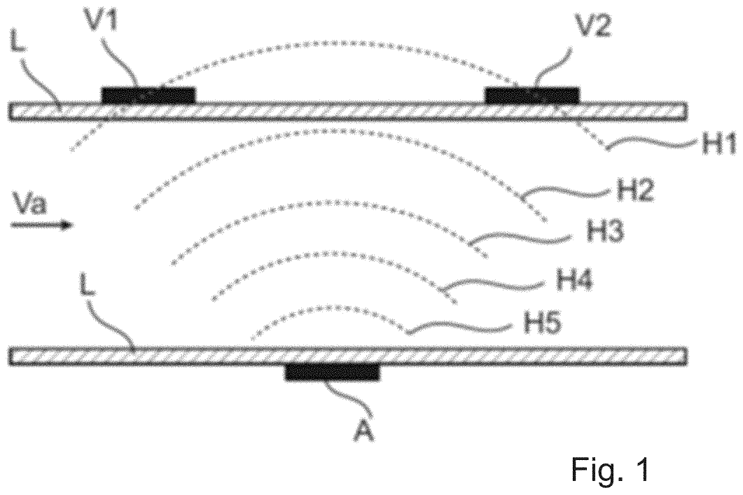

Turning initially to FIG. 1, there is shown a schematic of the core operational aspects of the invention. There is provided a conduit L in longitudinal section with a flow medium streaming with a flow rate Va. The conduit may be configured to have a circular or angular, symmetrical or asymmetrical, flat or oblate cross section at the level of the measurement area. The cross-sectional area should be generally constant along the longitudinal direction of the conduit, but in some applications, it may be decreased in order to increase the flow speed and therefore increase the resolution and the accuracy of the measurement.

The conduit is provided on the outer surface with an acoustic transducer configured as a transmitter A for propagating an acoustic compression wave which radiates from the source along two diagonal transmission paths through the streaming medium in an upstream and a downstream direction to the two receiving transducers V1 and V2. V1 and V2 are configured as receiving transducers and positioned on the outer surface of the conduit and opposite to transmitter A. The two receivers V1 and V2 are located in a diagonal upstream and downstream position relative to the transmitter A. The receivers V1 and V2 may be placed symmetrical or asymmetrical to the transmitter A. The transmitter A and the receivers V1 and V2 may be piezoelectric devices clamped on the outer surface of the conduit wall for generating and receiving ultrasonic waves. Transducers used as a transmitter or a receiver may have an identical or similar construction, however one of the transducers is configured to be used always as a transmitter and two other transducers are configured to be used always as receivers. If the ultrasonic flow signals are combined with the pressure signal, it is possible to carry out a continuous measurement without any interruption necessary to change the direction of transmission as is typical in prior art systems.

V1 and V2 receivers are positioned so that they are irradiated by the transmitter. The sensitivity of the transducers configured to be used always as receivers can have a much higher level than that of the prior art systems where the transducers were used alternatively as a transmitter and a receiver.

Transmitter A emits a compression wave in the form of a pulse train, with pulses H1, H2, H3, H4, H5 and so forth. The longitudinal waves propagate semi-spherically towards the receivers in the order they were emitted and upon arrival they excite the receivers.

The transmitter shown in FIG. 1 has a wide radiation angle in order to irradiate both receivers. In order to provide for a sufficient level of excitation in the receivers, the transmitter has to transmit longitudinal waves with a relatively high power.

The transmitter A, is a wide angle radiator and the receivers V1 and V2 are located within a range irradiated by the transmitter. In order to provide waves emitted from the piezoelectric devices in phase so that the different waves do not interfere with each other resulting in a decrease of the amplitude, the piezoelectric devices generally are provided with a wear plate of a thickness of λ/4. In order to maintain this wave emitting characteristic of the piezoelectric devices, the overall thickness of such a wear plate and the wall of the conduit shall be preferably selected to be substantially λ/4. In one embodiment, this may be achieved by extenuating, removing or dimensioning the wear plate and selecting the wall thickness accordingly. In some embodiments, the wall thickness is selected to be thin and elastic enough to vibrate when the transducers are excited and oscillate. In order to minimize energy loss while transmitting the longitudinal waves through the conduit wall, the wall is preferably acoustical wave impedance coupled to the streaming medium.

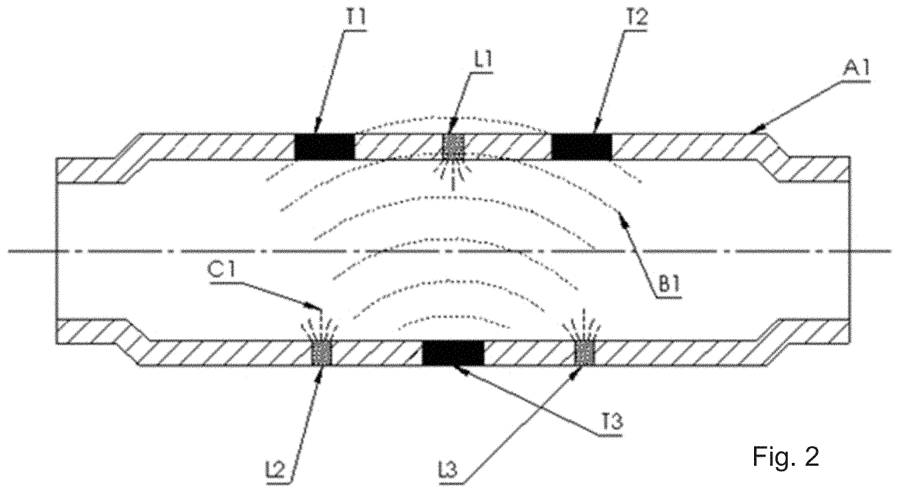

Turning now to FIG. 2, there is shown a further alternative ultrasonic flow sensor, including integrated with one or more pressure sensors. L1, L2 and L3. The pressure sensors can be pressure sampling tubes placed at various locations in relation to the T1, T2 and T3 flow transducers. A first solution is to implement a mirror symmetrical configuration where the pressure sensor L1 is located opposite to transducer T3. Another practical solution is to locate the pressure sampling tube at one end of the flow tube, to obtain the pressure input at a close location to the patient or simulated patient.

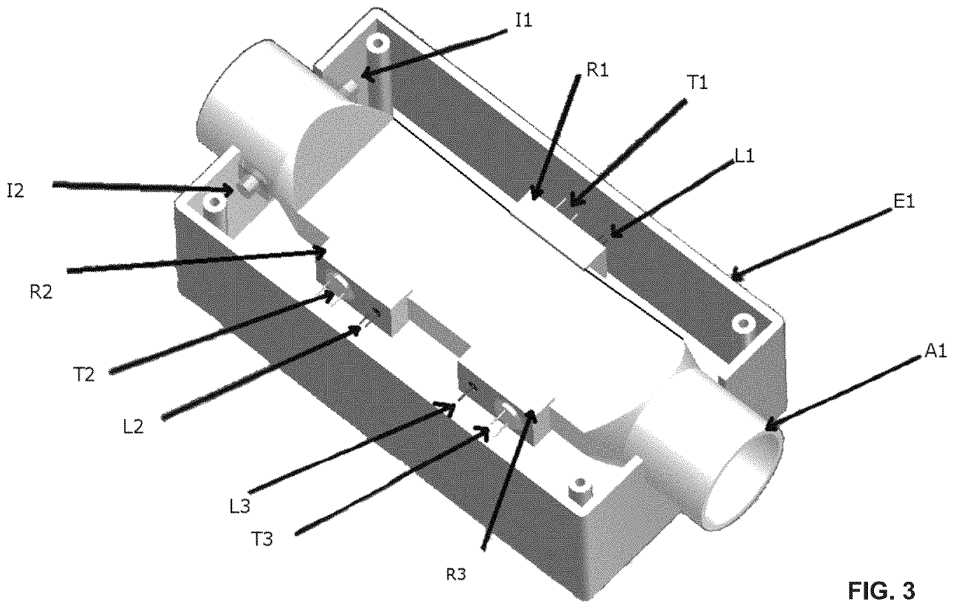

Turning now to FIG. 3, there is shown one modelled configuration where pressure outlets 11 and 12 are located at the very end of the tube for the above explained reason. Transducers T1, T2 and T3 are as previously specified, as are pressure sensors L1, L2 and L3, each located in a corresponding recess R1, R2, R3.

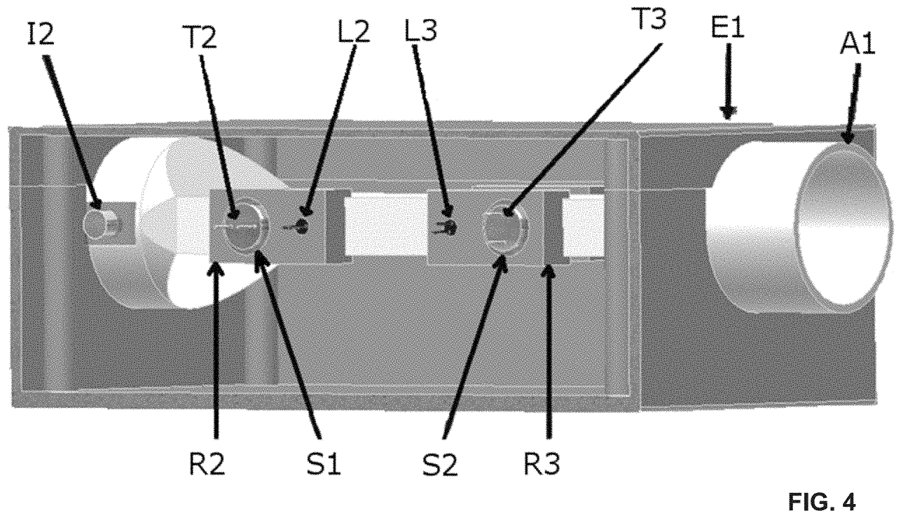

FIG. 4 demonstrates the relationship of the pressure sensor 12 to the flow and pressure sensors T2 and T3, and L2 and L3.

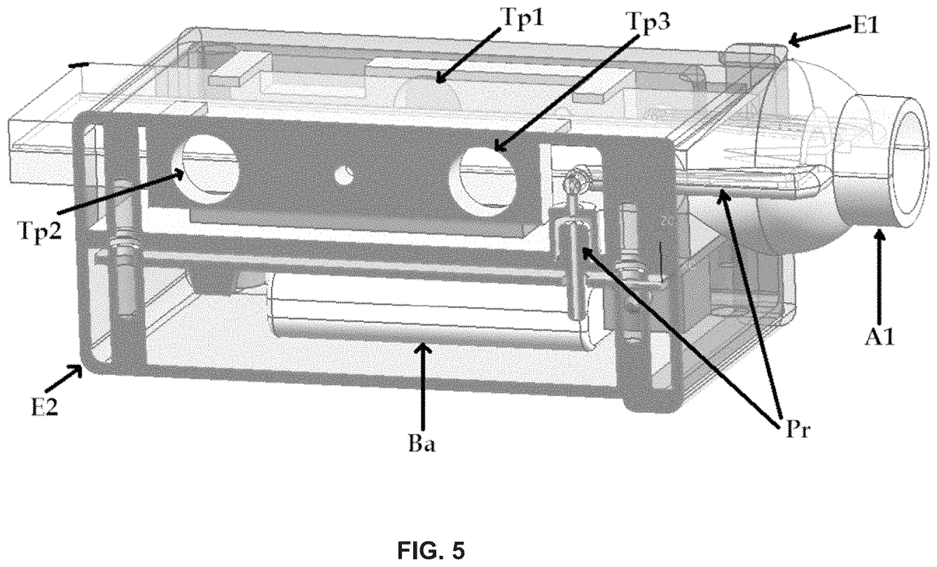

FIG. 5 illustrates a CAD output of a further alternative embodiment, where the measurement tube and the handheld device are detachable and the flow and volume data signal is transmitted via a connector. The pressure signal is transmitted via Pr sealed connectable tube configuration.

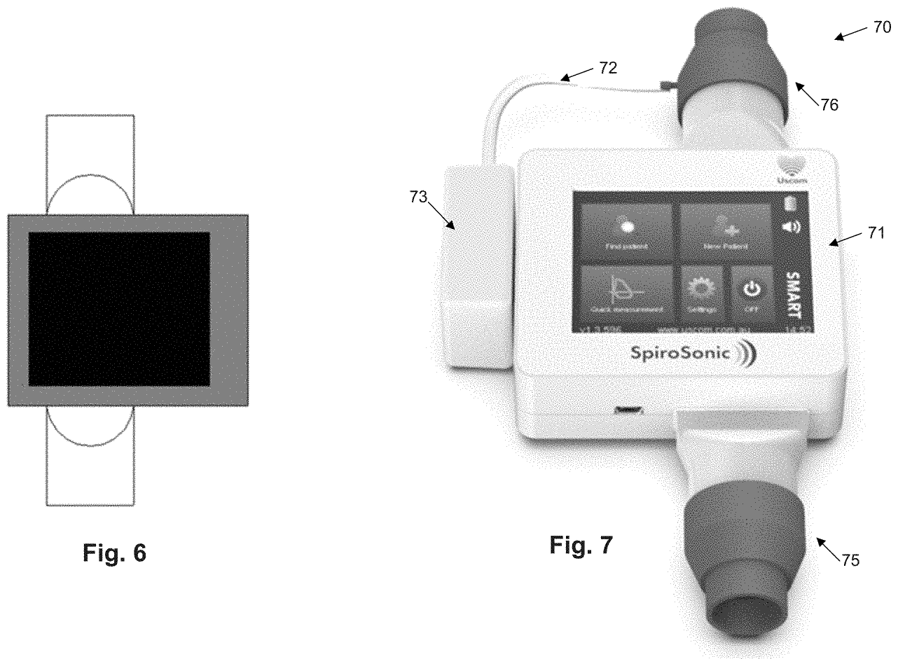

FIG. 6. is a simplified schematic view of another embodiment of the invention, a medical device, which can also be used as a spirometer with a self contained touchscreen display. The spirometer design can be upgraded to function as a medical ventilator tester.

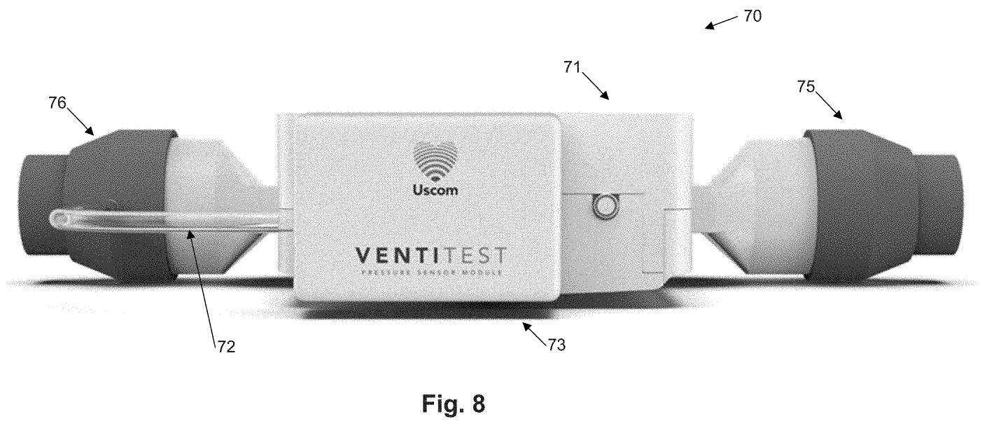

FIG. 7 and FIG. 8 is a is a detailed image of one form of CAD rendering of a final product 70. A medical device core framework system (spirometer) 71 is extended with a clamp-on pressure box 73. The flow tube is narrowed down to a standard 22 mm flow tube at both ends 76, 75, for easy connection to a medical ventilator. The pressure measurement subsystem 73 connects to the flow tube via a pressure sampling tube 72 and the data is transferred to the handheld device by means of a standard communications port. The device features a rechargeable battery as well as a touch screen display and firmware/software with an ergonomic user interface to display flow, volume and pressure basic values.

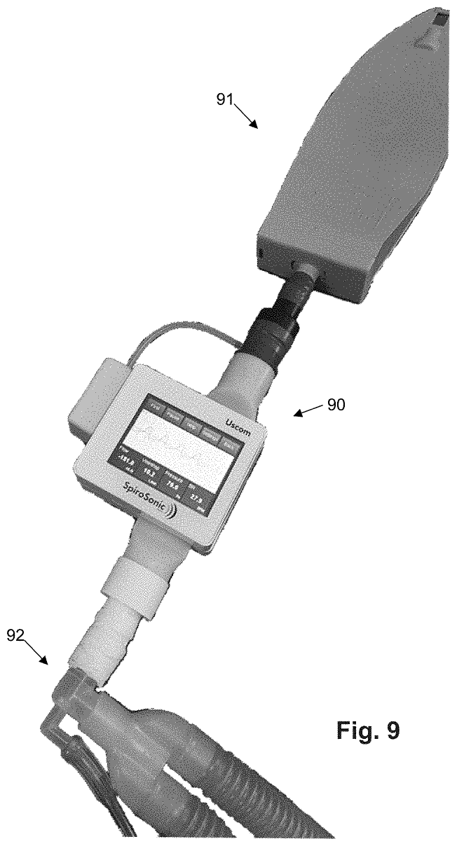

FIG. 9 demonstrates the device 90 connected to a test lung device 91 and ventilator patient circle 92 for measurement of air flows.

The embodiments provide for the examination, testing and intervention in the functionality of ventilators and other mechanical respiratory devices, including the following structure: a transducer (transmitter) generates longitudinal waves inside a flow tube, the waves are received by two transducers located diagonally to the transmitter on the opposite side of the flow tube (receivers). They can be placed on the wall of the flow tube. A pressure sensor, in a way so that the tube of the pressure sensor passes through the wall of the tube, stops in line with the plane of the inner surface, and the actual pressure sensor is located outside the wall of the tube. The resultant flow values and other parameters and characteristic of the measured flow medium can be determined from the measured values of the longitudinal waves. In some arrangements, the pressure sensor is preferably located exactly halfway between the two receivers, exactly opposite the transmitter.

Controller Architecture

The transducers and pressure sensors are interconnected to a monitoring system for the continuous monitoring of pressure flows within the tube. The monitoring system can take many different forms depending on the incorporation of relevant technology and requirements.

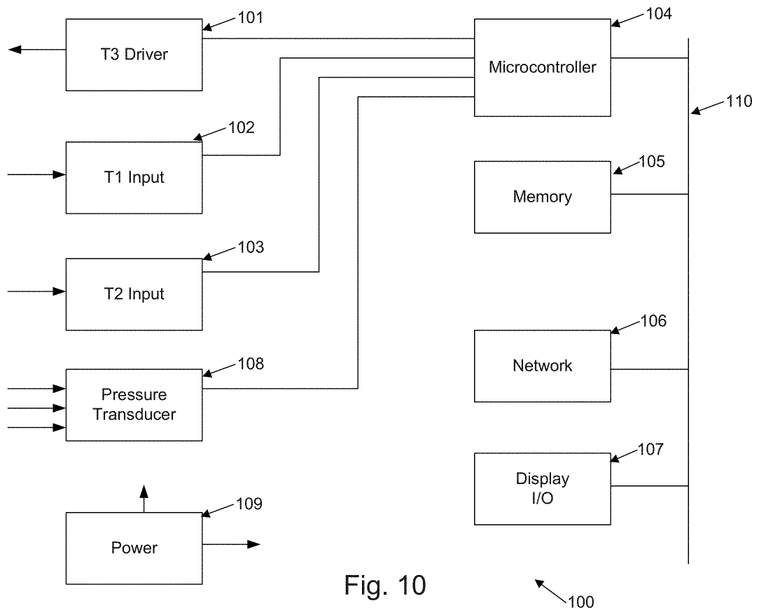

One form of monitoring system will now be described with reference to FIG. 10. In this arrangement of a monitoring system 100, a first transducer output unit 101 provides and output signal for driving transducer T3. Two transducer signal sampling units 102, 103, continuously sample the outputs from transducers T1 and T2. Further, optional pressure sampling unit 108 samples the pressure outputs P1 to P3. Each of these units is interconnected to a microcontroller 104 for download of the sampling streams and output of the T3 signal.

The microcontroller is programmed via software stored in memory 105, via bus 110. Bus 110 also connects a wireless communications driver chip 106 for wireless communications and a display and I/O unit for display of information and input of user input.

The microcontroller or other processor 104 is programmed to output a transducer control signal and sample transducer and pressure transducer outputs. Many different software architectures can be used for programming the microcontroller to eject transducer signals into the cavity and sample the responses via transducers T1 and T2.

The flow and volume calculations are processed in accordance with the techniques mentioned in the earlier mentioned PCT application PCT/HU2008/000146.

The pressure signal is measured by the means of an ND converter, and the digital data is merged with the flow and volume information in the microcontroller (which can also include a microprocessor, FPGA, CPLD or other processor).

The zero crossing of the flow data is matched by the zero crossing of the pressure signal; the synchronized signals together provide a more accurate measurement of flow and volume, especially in case of longer term measurements.

The calculated volume and the synchronized pressure-flow information is transmitted real-time or near real-time to the computer or a display device. In a preferred embodiment the communication frequency is 100 Hz. The display device can be a robust handheld monitor for use in the field.

The flow, volume and pressure information is displayed on a graphical user interface and further parameters are calculated and displayed in time, including time parameters and dynamic and absolute minimum, maximum and average values.

Many different schema can be used for transmitting the pressure waves. The longitudinal waves can be ultrasonic waves generated by a piezoelectric device. The longitudinal waves can be generated by a transducer used as a transmitter and can be in the form of wave packages separated from each other by a period sufficiently long for identifying the appropriate pulse packages. Subsequent wave packages following each other can be shifted in phase with respect to each other wherein the phase shift is selected randomly between a minimum and a maximum value, for inhibiting the forming of standing waves inside the conduit.

In some embodiments, the transit time is determined by measuring the time between a selected point of the transmitted wave and a corresponding selected point of the received wave. The selected point of the received wave can be determined by comparing the received wave with a reference signal of a predetermined level being above the noise level. The selected point of the received wave can be determined as a first zero crossing after the signal level exceeded the comparator level.

The selected point of the transmitted and received wave can be determined as a zero crossing of a selected rising edge of the respective signal. The transit time of the waves between the transducer used as a transmitter and the transducers used as receivers can be determined by: measuring the transit time of subsequent waves and generating an average value of several transit time values.

The transit time can be determined by determining a transit time between the transducer used as a transmitter and a transducer used as receivers under normal conditions when the flow rate is zero; measuring a phase shift of the zero crossing of a corresponding rising edge of the received signal; calculating a time difference corresponding to the phase shifting, and adding the time difference to the transit time under zero flow condition.

The time difference can be determined by: measuring a time difference for subsequent zero crossings in the received wave and generating an average value of several time differences. The zero crossing can be used for determining the time difference when the amplitude of the received signal has exceeded a predetermined comparator level.

In some embodiments, a zero crossing is used for determining the time difference when the zero crossing is inside a time window determined by minimum and maximum streaming conditions. The time window can be determined by a gating signal having a rising edge at the beginning of the time window and a falling edge at the end of the time window. The gating signal can be selected so that it starts after the transversal component of the wave propagating in the wall of the tube has reached the receivers and it ends before significant reflected waves arrive at the receivers.

The transit time can be determined in case of a phase jump of the zero crossing in the received wave by adding or subtracting a compensating value to the time difference corresponding to a total wave of the received signal. Phase jumps can also be filtered out with low pass filters.

The transducers used as receivers are controlled to minimize their sensitivity in a time interval outside the time window for receiving the waves transmitted by the transducer used as a transmitter.

The transit times between the transducer used as a transmitter and the transducers used as receivers can be determined under zero flow condition wherein the transducers used as receivers are located symmetrical relative to the transducer used as a transmitter and if a difference between the two transit times is detected, an offset value is determined and all subsequent measured values are corrected on the basis of the offset value.

The transit times between the transducer used as a transmitter and the transducers used as receivers are determined under zero flow conditions, wherein the transducers used as receivers are located asymmetrical relative to the transducer used as a transmitter and if a difference between a calculated or nominal position and an actual position of the transducer used as a transmitter can be detected, a correction value is determined and all subsequent measured values are modified with the correction value.

Some embodiments therefore provide a method and apparatus, for the examination, testing and intervention in the functionality of ventilators and other mechanical respiratory devices, including the following structure: a transducer (transmitter) generates longitudinal waves inside a flow tube, the waves are received by two transducers located diagonally to the transmitter on the opposite side of the flow tube (receivers), a pressure sensor is placed on the wall of the flow tube in a way so that the tube of the pressure sensor passes through the wall of the tube, stops in line with the plane of the inner surface, and the actual pressure sensor is located outside the wall of the tube.

The flow and volume values and other parameters characteristic of the measured flow medium are determined from the measured values of the longitudinal waves. The pressure sensor is preferably located exactly halfway between the two receivers, exactly opposite the transmitter.

The conduit used in the apparatus comprises a first location for receiving a transmitter in a middle region of the measuring area and two second locations for receiving receivers in a border region of the measuring area opposite to the first location. The wall of the conduit is dimensioned so that the longitudinal waves can pass through it with minimal loss and maximum efficiency or the transducers are sunk in the wall.

The inner wall of the conduit forms a uniform and continuous surface for the transmission of the longitudinal waves between the transmitter and the receivers and for blocking the passage of any organic or inorganic material.

In an embodiment the conduit connects to a medical ventilator via an industry standard connector tube and a very low flow resistance/obstacle. In such an embodiment of the invention the apparatus is connected to a medical ventilator in a way that the flow, volume and pressure signals are used in a direct feedback to the ventilator and the measured precision values are used to set the corresponding offset parameters.

FIG. 6. is a simplified schematic view of an embodiment of the invention, a medical device, which can also be used as a spirometer. In turn, the spirometer design can be applied as a basis to extend the system functionality to operate as a medical ventilator tester.

FIG. 7 and FIG. 8 are more detailed images of an embodiment of the invention. In this embodiment, the medical device core system (spirometer) based on the aforementioned PCT application, is extended with a clamp-on pressure box 73, the flow tube is narrowed down to a standard 22 mm flow tube at both ends for easy connection to a medical ventilator. The pressure measurement subsystem connects to the flow tube via a thin pressure signal tube and the data is transferred to the handheld device by means of a standard communications port. The device features a rechargeable battery as well as a touch screen display and a firmware with an ergonomic user interface to display flow, volume and pressure basic values.

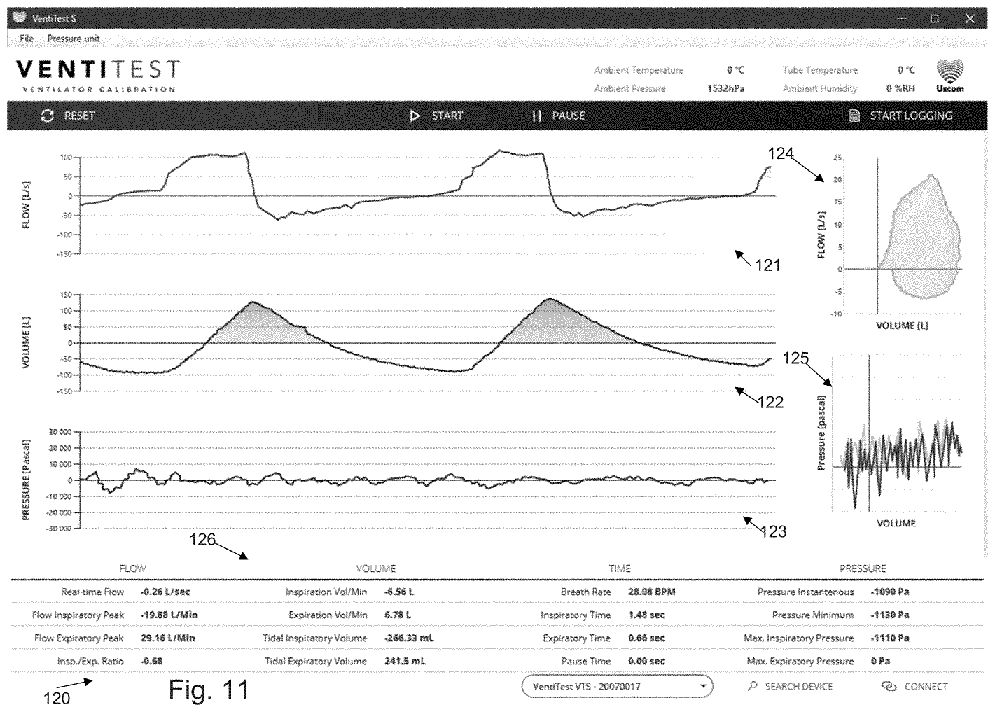

FIG. 11 is an example user interface 120 illustrating real time plots of flow 121, volume 122 and pressure 123. Various other plots can be shown in real time, including volume versus flow 124 and pressure versus volume 125. Also, shown are real time numerical values for various parameters 126. The information output can be subject to real time update at predetermined intervals.

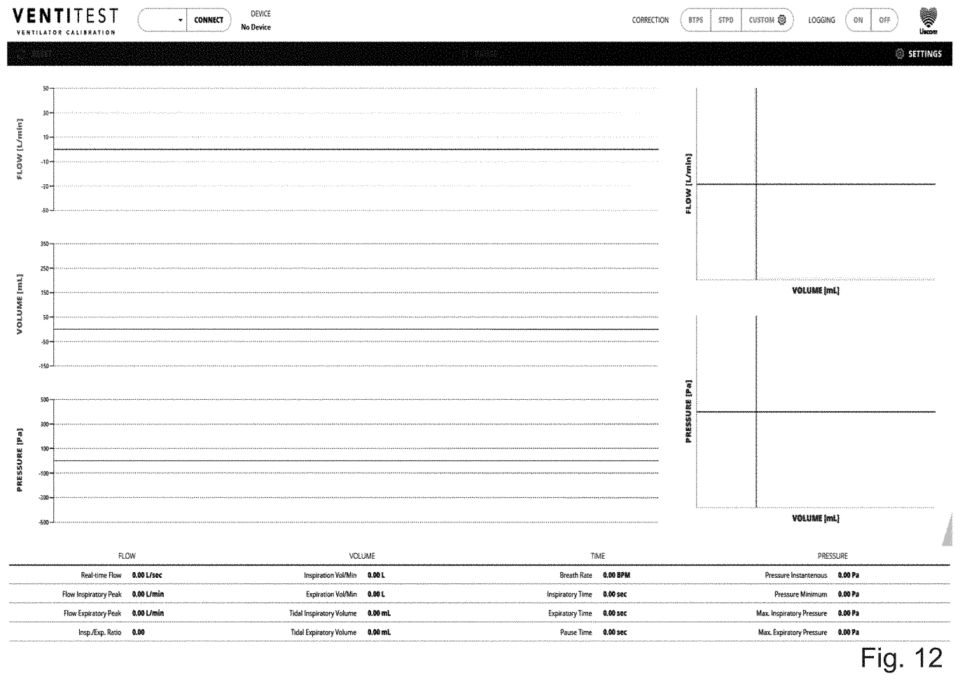

FIG. 12 is a prototype user interface of another form of display of the flow, volume and the pressure signals, on separate graphs, with advanced calculated parameters, which can be used for direct comparison with the preset ventilator parameters.

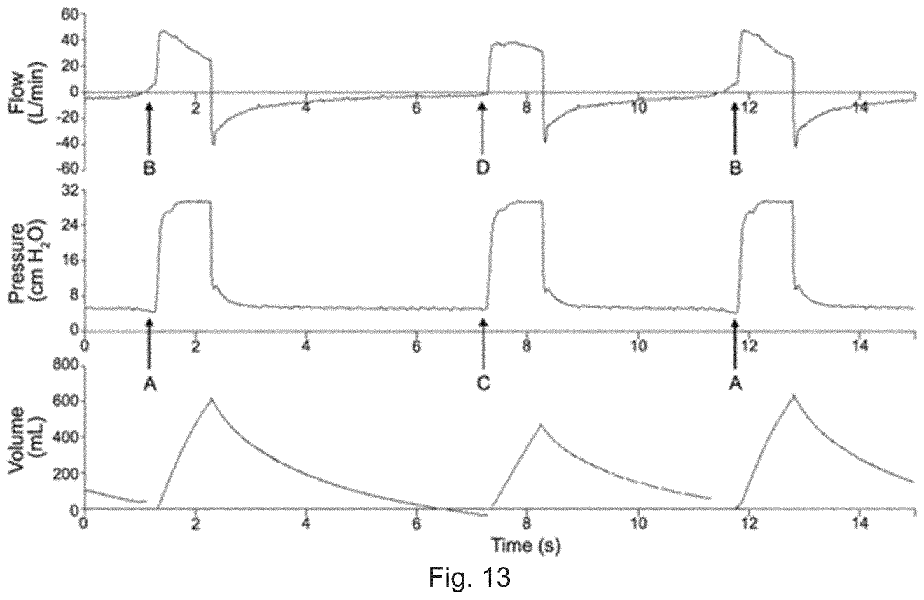

FIG. 13 shows the synchronization of the flow and pressure signals to calculate a very accurate volumetric data. In a calibration, the timing of the flow zero-crossing (B, D) is identical with the timing of the pressure signal trigger (A,C)., and the volume integration calculation from flow and time starts in B and D triggers. However, in real life implementation, we have experienced a “hovering effect”, that is, the flow zero-crossing does not correspond with the pressure trigger. The volume therefore does not return to zero after an inhale-to-exhale cycle and it tends to increase or decrease by time. To eliminate this effect, we use the pressure trigger signal to adjust the flow value after each respiratory cycle, hence the flow and the pressure zero triggers will be synchronized and any hovering effect will be eliminated in consecutive cycles. This co-processing of the sensor signals greatly increases the long term monitoring accuracy of the ventilator tester.

### Interpretation

Reference throughout this specification to “one embodiment”, “some embodiments” or “an embodiment” means that a particular feature, structure or characteristic described in connection with the embodiment is included in at least one embodiment of the present invention. Thus, appearances of the phrases “in one embodiment”, “in some embodiments” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

As used herein, unless otherwise specified the use of the ordinal adjectives “first”, “second”, “third”, etc., to describe a common object, merely indicate that different instances of like objects are being referred to, and are not intended to imply that the objects so described must be in a given sequence, either temporally, spatially, in ranking, or in any other manner.

In the claims below and the description herein, any one of the terms comprising, comprised of or which comprises is an open term that means including at least the elements/features that follow, but not excluding others. Thus, the term comprising, when used in the claims, should not be interpreted as being limitative to the means or elements or steps listed thereafter. For example, the scope of the expression a device comprising A and B should not be limited to devices consisting only of elements A and B. Any one of the terms including or which includes or that includes as used herein is also an open term that also means including at least the elements/features that follow the term, but not excluding others. Thus, including is synonymous with and means comprising.

As used herein, the term “exemplary” is used in the sense of providing examples, as opposed to indicating quality. That is, an “exemplary embodiment” is an embodiment provided as an example, as opposed to necessarily being an embodiment of exemplary quality.

It should be appreciated that in the above description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

Furthermore, some of the embodiments are described herein as a method or combination of elements of a method that can be implemented by a processor of a computer system or by other means of carrying out the function. Thus, a processor with the necessary instructions for carrying out such a method or element of a method forms a means for carrying out the method or element of a method. Furthermore, an element described herein of an apparatus embodiment is an example of a means for carrying out the function performed by the element for the purpose of carrying out the invention.

In the description provided herein, numerous specific details are set forth. However, it is understood that embodiments of the invention may be practiced without these specific details. In other instances, well-known methods, structures and techniques have not been shown in detail in order not to obscure an understanding of this description.

Similarly, it is to be noticed that the term coupled, when used in the claims, should not be interpreted as being limited to direct connections only. The terms “coupled” and “connected,” along with their derivatives, may be used. It should be understood that these terms are not intended as synonyms for each other. Thus, the scope of the expression a device A coupled to a device B should not be limited to devices or systems wherein an output of device A is directly connected to an input of device B. It means that there exists a path between an output of A and an input of B which may be a path including other devices or means. “Coupled” may mean that two or more elements are either in direct physical or electrical contact, or that two or more elements are not in direct contact with each other but yet still co-operate or interact with each other.

Thus, while there has been described what are believed to be the preferred embodiments of the invention, those skilled in the art will recognize that other and further modifications may be made thereto without departing from the spirit of the invention, and it is intended to claim all such changes and modifications as falling within the scope of the invention. For example, any formulas given above are merely representative of procedures that may be used. Functionality may be added or deleted from the block diagrams and operations may be interchanged among functional blocks. Steps may be added or deleted to methods described within the scope of the present invention.

### LIST OF REFERENCES USED IN THE DRAWING

A, A_{B}, Ba1, Ba2 transmitter
V1, V2 receiver
L conduit
Va flow rate
H1-H5 longitudinal waves
t1, t2 transit time
t₁₁-t₁₄, t₂₁-t₂₄ transit time
P0, P1, P2 position
a, b distance
s, sx, sy, s1, s2 distance
Sz1-Sz3 section
AJ pulse train for exciting the transmitter
V1J, V2J signal of the receivers
Tw time window
Wr reference wave
T0 transit time for zero flow rate
Δt1-Δt5 time shift value
S0, S1, S2 received wave
t_{D} determined time shift value
t_{C} corrected time shift value
e1, e2 line
BB internal cover
BK external cover
AK streaming medium
I adjusting pin
R spring
Z1 latch
Z2 pin
B1, B2 part of house
ZS, Zs1 hinge
ES1, ES2 end section
MS middle section
OA transmitting area
OV1, OV2 receiving area
IL inner lining
RP head portion
BP body portion
HP handle portion
K housing
K1, K2 parts of the housing
OL opening
PT protrusion
G ball
S spring
c velocity of the ultrasound
SZ symmetry axis
tak starting time
tav ending time
T period
Δt transit time difference
Vol1, Vol2, Vol3, Vol21, Vol22—volume
10, 11 pin
12, 13 button
14 handle
15 (LCD) display
E1, E2 electrodes
Tr transducer
O opening
Wp wear plate
PC piezo crystal

1. A method of monitoring, detecting, or observing the flow and volume of a gas along a channel, the method comprising the steps of:
utilising at least a first ultrasonic transducer to project an alternating ultrasonic signal substantially transverse to the direction of gas flow;
sampling the ultrasonic signal after it traverses the gas flow;
utilising at least a first pressure sensor to simultaneously monitor the pressure variations within the tube; and
processing the sampled signal and pressure measurements to determine properties of the gas and flow parameters relating thereto.

2. A method as claimed in claim 1 wherein said sampling includes sampling the ultrasonic signal at least two points substantially opposite said first ultrasonic transducer.

3. A method as claimed in claim 2 wherein at least one of the points is upstream of the first ultrasonic transducer and one is down stream of the first ultrasonic transducer.

4. A method as claimed in claim 1 further comprising:
simultaneously monitoring the gas pressure within the channel.

5. A method as claimed in claim 4, wherein the gas pressure is monitored at multiple points along said channel.

6. A method as claimed in claim 5 wherein one of the points is opposite the first ultrasonic transducer opposite the channel.

7. A device for monitoring the flow of a gas along a tube, the device comprising:
a first tube having an inlet and outlet for connection to a gas source and a gas sink;
at least one ultrasonic transducer located on one side of the tube for projecting an ultrasonic signal into the tube substantially transverse to the gas flow in the tube;
at least two ultrasonic sensors located on an opposed side of the tube for monitoring the receipt of the ultrasonic signal on the opposed side of said tube;
at least one pressure sensor for measuring pressure values within said tube; and
processing means interconnected to the at least one ultrasonic transducer and said two ultrasonic sensors and at least one pressure sensor for determining flow parameters of the gas within said tube.

8. An apparatus, for the examination, testing and intervention in the functionality of ventilators and other mechanical respiratory devices, comprising:
a flow tube along which a gas to be measured flows;
an ultrasonic transducer (transmitter) on one side of the flow tube, generating longitudinal waves inside the flow tube,
at least two transducers located on an opposed side of the flow tube, the waves are received by the two transducers (receivers),
a pressure sensor on the wall of the flow tube in a way so that the tube of the pressure sensor passes through the wall of the tube, stops in line with the plane of the inner surface, and the actual pressure sensor is located outside the wall of the tube.

9. The apparatus as claimed in claim 8 further comprising a monitoring unit for monitoring the flow and pressure measurements and determining parameters therefrom.

10. The apparatus as claimed in claim 8 wherein the longitudinal waves are ultrasonic waves generated by a piezoelectric device.

11. The apparatus as claimed in claim 8 wherein the longitudinal waves generated by the transducer used as a transmitter are in the form of wave packages separated from each other by a period sufficiently long for identifying the appropriate pulse packages.

12. The apparatus of claim 11 wherein subsequent wave packages following each other are shifted in phase with respect to each other wherein the phase shift is selected randomly between a minimum and a maximum value, for inhibiting the forming of standing waves inside the conduit.

13. The apparatus of claim 9 the transit time is determined by measuring the time between a selected point of the transmitted wave and a corresponding selected point of the received wave.

14. The apparatus as claimed in claim 13 wherein the selected point of the received wave is determined by comparing the received wave with a reference signal of a predetermined level being above the noise level.

15. The apparatus as claimed in claim 14 wherein the selected point of the received wave is determined as a first zero crossing after the signal level exceeded the comparator level.

16. The apparatus as claimed in claim 14 wherein the selected point of the transmitted and received wave is determined as a zero crossing of a selected rising edge of the respective signal.

17. The apparatus as claimed in claim 13 wherein the transit time of the waves between the transducer used as a transmitter and the transducers used as receivers is determined by
measuring the transit time of subsequent waves, and
generating an average value of said several transit time values.

18. The apparatus as claimed in claim 8, wherein the transit time is determined by
determining a transit time between the transducer used as a transmitter and a transducer used as receivers under normal conditions when the flow rate is zero,
measuring a phase shift of the zero crossing of a corresponding rising edge of the received signal,
calculating a time difference corresponding to said phase shifting, and
adding the time difference to the transit time under zero flow condition.

19. The apparatus as claimed in claim 18, wherein the time difference is determined by
measuring a time difference for subsequent zero crossings in the received wave and
generating an average value of several time differences.

20. The apparatus as claimed in claim 18, wherein a zero crossing is used for determining the time difference when the amplitude of the received signal has exceeded a predetermined comparator level.

21. The apparatus as claimed in claim 18, wherein a zero crossing is used for determining the time difference when the zero crossing is inside a time window (gate wait) determined by minimum and maximum streaming conditions.

22. The apparatus as claimed in claim 21, wherein the time window will be determined by a gating signal having a rising edge at the beginning of the time window and a falling edge at the end of the time window.

23. The apparatus as claimed in claim 22, wherein the gating signal is selected so that it starts after the transversal component of the wave propagating in the wall of the tube has reached the receivers and it ends before significant reflected waves arrive at the receivers.

24. The apparatus as claimed in claim 18, wherein the transit time is determined in case of a phase jump of the zero crossing in the received wave by adding or subtracting a compensating value to the time difference corresponding to a total wave of the received signal.

25. The apparatus as claimed in claim 21, wherein the transducers used as receivers are controlled to minimize their sensitivity in a time interval outside the time window for receiving the waves transmitted by the transducer used as a transmitter.

26. The apparatus as claimed in any of claims 9 to 25, wherein the transit times between the transducer used as a transmitter and the transducers used as receivers are determined under zero flow condition wherein the transducers used as receivers are located symmetrical relative to the transducer used as a transmitter and if a difference between the two transit times is detected, an offset value is determined and all subsequent measured values are corrected on the basis of the offset value.

27. The apparatus as claimed in any of claim 9, wherein the transit times between the transducer used as a transmitter and the transducers used as receivers are determined under zero flow condition, wherein the transducers used as receivers are located asymmetrical relative to the transducer used as a transmitter and if a difference between a calculated or nominal position and an actual position of the transducer used as a transmitter can be detected, a correction value is determined and all subsequent measured values are modified with the correction value.
